(19) United States
(12) Patent Application Publication
(10) Pub. No.: US 2006/0188951 A1
(43) Pub. Date: Aug. 24, 2006

(54) Method for measuring the level of anti-beta-amyloid antibody in body fluids and diagnostic kit for alzheimer's disease using same

(76) Inventors: In Hee Mook, Seongnam-si Kyungki-do (KR); Ki Yeun Hur, Suwon-si, Kyungki-do (KR)

Correspondence Address: ANDERSON, KILL & OLICK, P.C., 1251 AVENUE OF THE AMERICAS, NEW YORK,, NY, 10020-1182, US

(21) Appl. No.: 10/546,547

(22) PCT Filed: Feb. 24, 2004
(86) PCT No.: PCT/KR04/00371

§ 371 Date: Aug. 23, 2005

(30) Foreign Application Priority Data
Feb. 24, 2003 (KR) 10-2003-0011440

(51) Int. Cl. G01N 33/537; C12M 1/34

(52) U.S. Cl. 435/7.92; 435/287.2

(57) ABSTRACT

A method for measuring a concentration of a β-amyloid antibody in a body fluid sample through a reaction forming an antigen-antibody complex uses an antigen protein binding specifically to the β-amyloid antibody existing in a body fluid of an Alzheimer's disease patient. A diagnostic kit for Alzheimer's disease using the method is also provided.

### FIELD OF THE INVENTION

The present invention relates to a method for measuring a concentration of a β-amyloid antibody in a body fluid sample through a reaction forming an antigen-antibody complex that uses an antigen protein binding specifically to the β-amyloid antibody existing in a body fluid of Alzheimer's disease patient and a diagnostic kit for Alzheimer's disease using the method.

### BACKGROUND OF THE INVENTION

Alzheimer's disease (AD) is one of the neurodegenerative diseases that occur as one gets older and is characterized by memory loss. As the aging population grows, so does the number of AD patients; and AD has presented itself as a serious social challenge due to its social and economical burdens.

As pathological characteristics of AD, senile plaques and neurofibrillary tangles in patients' brain have been observed, and owing to this, the loss of nerve cells has been clearly identified. Over 80% of a senile plaque consists of a toxic protein called β-amyloid (Aβ). Currently, this Aβ protein is considered as a major cause of AD. The Aβ protein consists of 40 or 42 amino acids cleaved from an amyloid precursor protein (APP) by β-secretase and γ-secretase. The main biological function of the Aβ protein has not yet been known. It is known, however, that the Aβ protein functions as a toxic material by inducing an inflammatory reaction of microglia and impeding a neural development.

AD has been clinically diagnosed by monitoring the change pattern of specific brain tissues (hippocampus, cortex, etc.) by means of magnetic resonance imaging (MRI) and single-photon emission computed tomography (SPECT) and the change pattern of behavior and perception of a subject by means of detailed questions about his condition, such as mini mental status examination (MMSE). In addition, AD can be diagnosed with high accuracy by measuring a concentration ratio between Aβ1-42 peptide and Aβ1-40 peptide in a subject's cerebrospinal fluid (CSF). However, since this diagnostic method requires a CSF isolated from an aged subject, it has not been used as a general diagnostic method for AD.

### SUMMARY OF THE INVENTION

Accordingly, it is an object of the present invention to provide a method for measuring a concentration of an Aβ antibody in a body fluid to diagnose AD in its early stage and a diagnostic kit for AD employing the method.

### BRIEF DESCRIPTION OF THE DRAWINGS

The above and other objects and features of the present invention will become apparent from the following description of the invention, when taken in conjunction with the accompanying drawings, in which:

FIG. 1 represents electron microscopic photographs of an Aβ protein in fibrillar or aggregated form prepared in accordance with the present invention;

FIG. 2 shows a diagram of concentration of an Aβ antibody in blood samples from AD patients and normal people measured by three groups of enzyme-linked immunosorbent assays using soluble, fibrillar, and aggregated Aβ1-42 antigens, respectively;

FIG. 3 depicts a diagram of measured concentration of the Aβ antibody according to FIG. 2, for those with ages ranging from 61 to 80;

FIG. 4 presents a diagram of concentration of an Aβ antibody in blood samples from cerebral apoplexy patients and normal people measured by three groups of enzyme-linked immunosorbent assays using soluble, fibrillar, and aggregated Aβ1-42 antigens, respectively; and

FIG. 5 offers a diagram of concentration of an Aβ antibody in blood samples from AD patients and normal people measured by an enzyme-linked immunosorbent assay using a soluble Aβ1-42 antigen under a different reaction condition.

### DETAILED DESCRIPTION OF THE INVENTION

The present invention provides a method for measuring a concentration of an Aβ antibody in a body fluid sample through a reaction forming an antigen-antibody complex that uses an antigen protein binding specifically to the Aβ antibody and a diagnostic kit for AD using the method.

The present inventors have found that the concentration of the Aβ antibody in body fluids of AD patients is different from that of normal people. The present invention, therefore, provides an in vitro analysis method for diagnosing AD by measuring the concentration of the Aβ antibody in a body fluid of an AD patient and comparing it with that of a normal person.

Specifically, the present invention provides a method for measuring a concentration of a β-amyloid antibody in a body fluid sample comprising the steps of: contacting an antigen specific to the β-amyloid antibody with body fluid samples from a test subject and a control subject in a reactor coated with the antigen; detecting the resulting antigen-antibody complexes by using a secondary antibody-label conjugate and a chromogenic substrate solution; and comparing the detected amount of the test subject with the detected amount of the control subject.

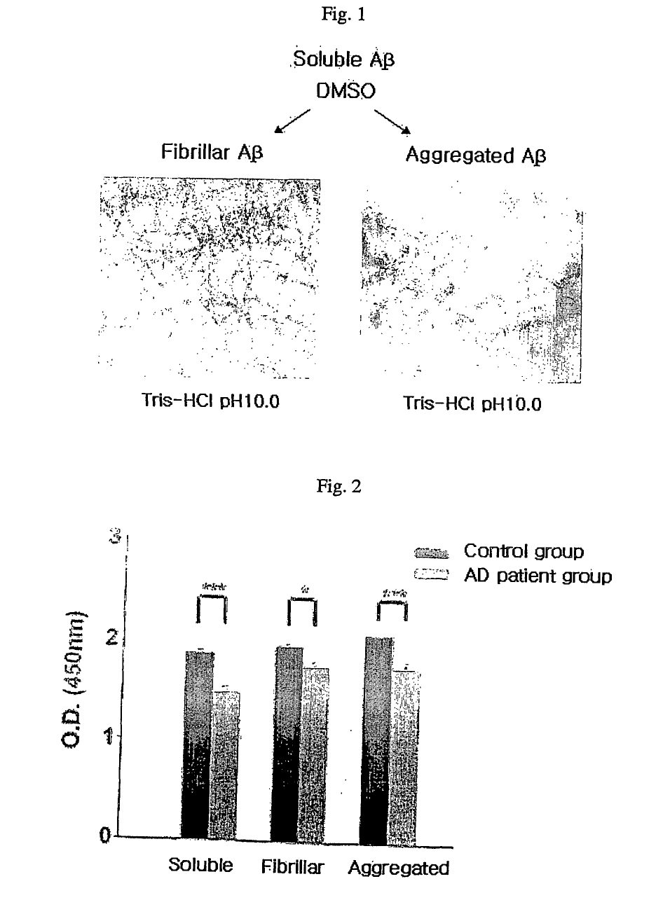

As shown in FIG. 1, an Aβ protein exists in a soluble, fibrillar or aggregated form in vivo.

To prepare the antigen protein specifically recognizing the Aβ antibody, an Aβ protein should be obtained. The Aβ protein may be synthesized based on a known amino acid sequence or produced in the form of a recombinant protein by genetic engineering. For example, a recombinant Aβ protein may be prepared by constructing an expression vector expressing the recombinant Aβ protein based on the nucleotide sequence of the Aβ gene as represented by SEQ ID NO:1, which is registered as GenBank Accession No. X06989 in NIH program GenBank; transforming an E. coli with the expression vector to obtain a transformant producing the recombinant Aβ protein; culturing the transformant; and isolating and purifying the recombinant Aβ protein.

The isolated and purified recombinant Aβ protein from the E. coli transformant or an Aβ (US Peptide Co.) synthesized by a peptide-manufacturing device based on the amino acid sequence of the Aβ protein of SEQ ID NO:2 is used as an antigen to measure the concentration of the Aβ antibody in a body fluid sample of a subject.

Since the Aβ protein exists in a soluble, fibrillar or aggregated form in vivo, the antigen protein specific to the Aβ antibody of the present invention may be prepared accordingly.

Specifically, the antigen protein may be Aβ1-42 represented by SEQ ID NO:3, an Aβ protein consisting of 42 amino acids, and may exist in a soluble, fibrillar or aggregated form under proper treatment. Besides, fragments of Aβ1-42 may be used as the antigen protein as long as they bind specifically to the Aβ antibody in a body fluid sample. For instance, Aβ1-40 peptide, Aβ25-35 peptide, etc., prepared by deleting a portion of amino acids from the Aβ1-42 of SEQ ID NO:3 can be used as the antigen protein.

Further, as for the antigen protein, a conventional protein used to improve the immunological effect can be conjugated to the whole Aβ1-42 or a fragment thereof. Specifically, the antigen protein may form a conjugate with streptoavidin or bovine serum albumin (BSA).

In the inventive method of measuring the concentration of the Aβ antibody, any body fluid samples obtained from any body regions can be used. It is, however, preferable to use blood considering its availability.

Any conventional immunological detection methods well known in the art using a secondary antibody-label conjugate and a chromogenic substrate solution, such as enzyme-linked immunosorbent assay (ELISA), radioimmunoassay (RIA), sandwich assay, western blotting on polyacrlamide gel, immunoblotting, immuno-fluorescence assay (IFA), immunochemiluminescence assay or immunochromatography (Rapid), may be used to detect an antigen-antibody complex resulting from a reaction between the antigen protein specific to the Aβ antibody and an antibody in a body fluid sample.

Then, the concentration of the Aβ antibody in a body fluid sample can be measured by comparing a detected amount of a test subject with that of a control subject.

Also, the present invention provides a protein chip for detecting the Aβ antibody in a body fluid sample of a test subject, which is prepared by fixing the antigen specific to the Aβ antibody on a biological microchip. Samples can be massively analyzed by employing the conventional biological microchip and automated microarray system as well as ELISA.

Further, the present invention provides a diagnostic kit for AD in its early stage, which comprises the antigen protein specific to the Aβ antibody.

The diagnostic kit of the present invention comprises:

a reactor coated with the antigen protein specific to the Aβ antibody;

a secondary antibody-label conjugate detecting the antigen-antibody complex and comprising a label performing a chromogenic reaction by reacting with a chromogenic substrate;

a chromogenic substrate solution comprising the chromogenic substrate;

a washing solution used in each reaction step; and

a solution for stopping the chromogenic reaction.

The diagnostic kit can diagnose AD by analyzing quantitatively and qualitatively the antigen protein for the Aβ antibody through a reaction forming an antigen-antibody complex. The antigen-antibody complex can be detected by the conventional ELISA, RIA, sandwich assay, western blotting on polyacrylamide gel, immunoblotting, immuno-fluorescence assay, immunochemiluminescence assay or immunochromatography. For example, a diagnostic kit can be prepared to perform ELISA using 96-well microtiter plates coated with the recombinant antigen protein.

It is preferred that the antigen protein coated on the reactor of the diagnostic kit be the antigen protein mentioned in the above method for measuring the concentration of the Aβ antibody in a body fluid sample.

The reactor coated with the antigen protein may be a nitrocellulose membrane, a 96 well plate made of polyvinyl resin, a 96 well plate made of polystyrene resin, and a slide glass.

As mentioned above, it is preferred to synthesize the antigen protein of the present invention with reference to a known amino acid sequence or prepare it in the form of a recombinant protein by genetic engineering. The antigen protein specific to the Aβ antibody may be adequately coated to compare the concentration of the Aβ antibody in an AD patient and a normal person without errors, and, preferably, in the range from 1 ng/100 μl to 100 μg/100 μl per a reactor.

Any conventional labels performing chromogenic reaction may be used as the label of the secondary antibody. These include horseradish peroxidase (HRP), alkaline phosphatase, colloidal gold, fluorescein such as poly L-lysine-fluorescein isothiocyanate (FITC) and rhodamine-B-isothiocyanate (RITC), and dye. In a preferred embodiment of the present invention, anti-human IgG-HRP conjugate is used.

The chromogenic substrate is determined depending on the label. Specifically, 3,3′,5,5′-tetramethyl bezidine (TMB), 2,2′-azino-bis(3-ethylbenzothiazoline-6-sulfonic acid) (ABTS) or o-phenylenediamine (OPD) may be used. It is preferred to dissolve the chromogenic substrate in a buffer solution (0.1M sodium acetate, pH 5.5). HRP, a label of the secondary antibody conjugate, decomposes the chromogenic substrate, such as TMB to generate a chromogenic precipitate. By checking the degree of the precipitation of the chromogenic precipitate with naked eye, the concentration of the Aβ antibody in a sample can be measured.

The washing solution preferably comprises a phosphate buffer solution, NaCl, and Tween 20, and, more preferably, 0.02M phosphate buffer solution, 0.13M NaCl, and 0.05% Tween 20. A proper amount of the washing solution is added to the reactor 3 to 6 times after the secondary antibody reacts with the antigen-antibody complex. A phosphate buffer solution containing 0.1% BSA may be used as a blocking solution, and 2N sulfuric acid solution may be added as a solution for stopping the chromogenic reaction.

The present inventors have investigated the distribution pattern of Aβ antibodies recognizing three different forms of in vivo Aβ (soluble, fibrillar, and aggregated) and their respective concentration in an AD patient group and a control group (normal people) in AD examination using the antigen protein specific to the Aβ antibody. As a result, Aβ antibodies recognizing the soluble, fibrillar, and aggregated Aβs were detected in blood samples of both the AD patient group, and the control group. But, it was observed that the measured concentration of the Aβ antibody from the blood samples in the AD patient group was significantly lower than that of the control group. To confirm whether the Aβ antibody is AD-specific antibody, the concentration of the Aβ antibody in blood samples from the cerebral apoplexy patient group and control group was measured using the method of the present invention, and the difference between the two groups was not significant. In addition, even when the blood sample was diluted in a ratio of 1:100 and anti-human IgG secondary antibody was diluted in a ratio of 1:2,000, it was possible to distinguish the AD patient group from the control group.

The above results clearly show that the method of the present invention can measure the concentration of the Aβ antibodies recognizing the soluble, fibrillar, or aggregated Aβ in patient's body fluid and can be a useful tool to diagnose AD easily and accurately.

Accordingly, the method for measuring the concentration of the Aβ antibody in accordance with the present invention and a diagnostic method or kit for AD employing the above method are new immunological diagnostic tools using the body fluid of a patient, and are capable of performing more accurate, reproducible, and convenient AD diagnosis in early stage than the conventional methods using MRI, SPECT, etc. or measuring the concentration ratio between Aβ1-42 peptide and Aβ1-40 peptide from patient's CSF.

The following Examples are intended to further illustrate the present invention without limiting its scope.

### EXAMPLE 1

### Preparation of an Antigen Specific to Aβ Antibody

To prepare an antigen specific to the Aβ antibody as means for diagnosing AD, a gene (GenBank accession No. X06989) for encoding β-amyloid consisting of 42 amino acids represented by SEQ ID NO:3, which is cleaved from APP by β-secretase and γ-secretase, is inserted into a multi-cloning site of pET-31b(+) vector (Novagen) to prepare pET-31b-Aβ1-42 vector. E. Coli BL21 (ATCC 47092) was transformed with the vector, and then 1 mM of isopropyl β-D-thiogalacto-pyranoside (IPTG; Sigma) was treated to induce the expression of the gene. A 55kDa Aβ1-42 fusion protein containing 40 kDa keto-steroid isomerase (KSI) was produced. The fusion protein was purified with His-Hind kit (Novagen). It was confirmed by immunoblotting that the purified recombinant protein contained the 55 kDa fusion protein as a major portion. The purified recombinant Aβ1-42 was used as an antigen in measuring the concentration of the Aβ antibody in a body fluid sample.

Alternatively, synthesized Aβ1-42 (US Peptide or Bachem) was used as an antigen in measuring the concentration of the Aβ antibody in a body fluid sample.

### EXAMPLE 2

### ELISA using an Antigen Specific to the Aβ Antibody

A method for measuring the concentration of the Aβ antibody in a blood sample was performed by ELISA using the antigen specific to the Aβ antibody in the following steps: 1) coating the well of ELISA plate with the Aβ antigen protein; 2) treating the well with a sample from a test subject and a control subject to react with the antigen protein coated on the well; and 3) measuring the concentration of the Aβ antibody in the samples from the test subject and the control subject and comparing the measured results.

<2-1> Preparation and Coating of a Structure-Specific Aβ Antigen Protein

A soluble Aβ protein was prepared by dissolving in dimethylsulfoxide (DMSO) 1 mg of the recombinant Aβ1-42 specific to the Aβ antibody or the Aβ1-42 synthesized by peptide-manufacturing device as described in Example 1. A fibrillar Aβ protein was prepared by dissolving 1 mg of the antigen protein Aβ1-42 in 50 μl of DMSO and adding thereto 950 μl of 1M Tris-HCl solution (pH 10.0). An aggregated Aβ protein was prepared by dissolving 1 mg of the antigen protein Aβ1-42 in 50 μl of DMSO and adding thereto 950 μl of phosphate buffered saline (PBS, pH 8.0).

The respective soluble, fibrillar, and aggregated Aβ antigen proteins were diluted with 0.1 M sodium bicarbonate buffer solution (pH 9.6, sigma), applied to 96-well ELISA plate (Maxisorp, Nunc) at a concentration of 0.1 μg/well, and then incubated overnight at 4° C. under a cover for coating.

Next, the well of the plate was rinsed three times with washing buffer solution (PBS containing. 0.05% Tween 20 (PBST), pH 7.4). and treated with 300 μl of blocking solution (PBST including 3% BSA, pH 7.4) for 2 hours at room temperature to block nonspecific protein binding site. Lastly, the blocking solution was removed, and the plate was vacuum-packed to be kept at 4° C.

<2-2> Reaction between Antigen Protein Coated on Well and Test Sample

Samples to be tested were prepared by isolating serum from the blood obtained from 46 AD patients, 13 cerebral apoplexy patients, and 228 normal people. The blood samples from the AD patients and the normal people were obtained from Samsung hospital in Seoul, Korea, and those from the cerebral apoplexy patients not showing the symptom of dementia were obtained from Aju University Hospital in Suwon, Korea. The age of the patients and the normal people (control group) ranged between 38 and 80.

100 μl of the blood samples diluted with PBS (1×) in a ratio of 1:10 was added to each of the wells coated with the soluble, fibrillar, and aggregated Aβ antigen proteins prepared in Example <2-1>. The wells were treated for 2 hours at 37° C. and then rinsed 4 times with washing buffer solution.

<2-3> Detection of Antigen-Antibody Complex

Anti-human IgG secondary antibody labelled with a horseradish peroxidase (Amersham Pharmacia Biotech) was diluted with PBST in a ratio of 1:1,000. Each well of the plate was treated with 100 μl of the diluted anti-human IgG secondary antibody for 1 hour at room temperature, and rinsed 3 times with washing buffer solution. Next, a chromogenic substrate solution was prepared by dissolving 1 mg of a chromogenic substrate TMB (Sigma Co., USA) in 10 ml of buffer solution (citric acid buffer solution, pH 5.0) and adding thereto 2 μl of 35% hydrogen peroxide. Each well was treated with 100 μl of the chromogenic substrate solution for 15 minutes at room temperature without light, and then the reaction was stopped by adding 50 μl of 2N sulfuric acid. The degree of chromogenic reaction was determined by measuring absorbance at a wavelength of 450 nm. All statistical analyses were performed by one-way analysis of variance (ANOVA) test. The respective results for the AD patients, the cerebral apoplexy patients, and the normal people are shown in FIGS. 2 to 4.

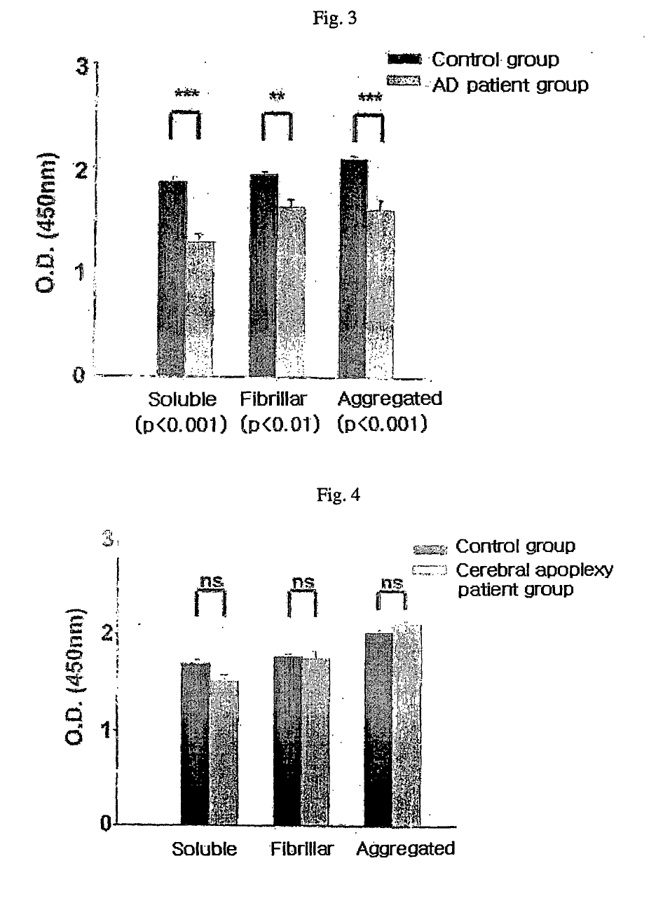

As shown in FIGS. 2 and 3, the absorbance of the AD patient group was significantly lower than that of the control group with regard to each well coated with the soluble, fibrillar, or aggregated Aβ antigen protein (soluble Aβ p<0.001; fibrillar Aβ p<0.05; and aggregated Aβ p<0.001) (FIG. 2). If the above results are reanalyzed by limiting the subject's age to those over 60, the difference is clearer (FIG. 3).

FIG. 4 shows concentration of the Aβ antibody in the blood samples of cerebral apoplexy patients (mean age: 64.7±11.6 years) and normal people (mean age: 56.2±10.1 years) measured by ELISA using the soluble, fibrillar, or aggregated Aβ31-42 antigen, to confirm that the concentration of the Aβ antibody significantly lower than that of normal people is caused by AD. As a result, unlike the result of the AD patients and the normal people, the concentration difference of the antibody recognizing the soluble, fibrillar, and aggregated Aβ was not significant between the cerebral apoplexy patients and the normal people (FIG. 4).

### EXAMPLE 3

### Measurement of the Concentration of the Aβ Antibody Under Different ELISA Reaction Condition

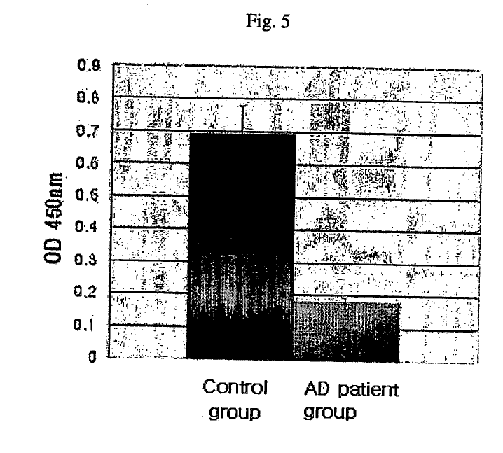

Except reaction conditions expressly described below, an experiment was performed according to the procedure described in Example 2 to measure an absorbance, and its results are illustrated in FIG. 5:

in Example <2-1>, a soluble Aβ protein was used as an antigen protein specific to the Aβ antibody, and a PBST containing 10% fetal bovine serum (FBS) was treated for one hour as a blocking solution instead of PBST containing 3% BSA;

in Example <2-2>, the blood samples were obtained from 11 normal people (control group) and 11 AD patients and diluted in a ratio of 1:100, instead of 1:10, and each well was rinsed 6 times with washing buffer solution;

in Example <2-3>, each well was treated with anti-human IgG secondary antibody diluted in a ratio of 1:2,000, instead of 1:1,000 at 37° C. for 30 minutes, and rinsed 6 times with washing buffer solution.

As shown in the graph of FIG. 5, even if the respective concentrations of the blood sample and the anti-human IgG secondary antibody are diluted 10 times and twice, in view of Example 2, the mean absorbance of the AD patients group was significantly lower than that of the normal people. Accordingly, it is clearly shown that AD patients can be identified even at a low blood concentration in accordance with the method of the present invention.

It is concluded that if the absorbance value for the Aβ antibody recognizing the soluble, fibrillar, or aggregated Aβ in a blood sample is relatively lower than that of normal people, the donor of the blood sample may be diagnosed as a potential AD patient.

While the invention has been described with respect to the above specific embodiments, it should be recognized that various modifications and changes may be made to the invention by those skilled in the art which also fall within the scope of the invention as defined by the appended claims.

What is claimed is:

1. A method for measuring a concentration of a β-amyloid antibody in a body fluid sample comprising the steps of:
contacting an antigen specific to the β-amyloid antibody with body fluid samples from a test subject and a control subject in a reactor coated with the antigen;
detecting the resulting antigen-antibody complexes by using a secondary antibody-label conjugate and a chromogenic substrate solution; and
comparing the detected amount of the test subject with the detected amount of the control subject.

2. The method of claim 1, wherein the antigen is of a soluble, fibrillar or aggregated β-amyloid.

3. The method of claim 1, wherein the antigen is a fragment of Aβ1-42 protein represented by SEQ ID NO:3.

4. The method of claim 3, wherein the fragment of Aβ1-42 protein is Aβ1-40 peptide or Aβ25-35 peptide.

5. The method of claim 1, wherein the antigen forms a conjugate with streptoavidin or bovine serum albumin.

6. The method of claim 1, wherein the antigen is coated at a concentration in the range from 1 ng/100 μl to 100 μg/100 μl per the reactor.

7. The method of claim 1, wherein the reactor is selected from the group consisting of a nitrocellulose membrane, a well plate made of polyvinyl resin, a well plate made of polystyrene resin, and a slide glass.

8. The method of claim 1, wherein the body fluid sample is blood.

9. The method of claim 1, wherein the secondary antibody-label conjugate comprises a label selected from the group consisting of horseradish peroxidase, alkaline phosphatase, colloidal gold, fluorescein and dye.

10. The method of claim 1, wherein the chromogenic substrate solution comprises a chromogenic substrate selected from the group consisting of 3,3′,5,5′-tetramethyl bezidine, 2,2′-azino-bis(3-ethylbenzothiazoline-6-sulfonic acid), and o-phenylenediamine.

11. The method of claim 1, wherein the resulting antigen-antibody complexes are detected by enzyme-linked immunosorbent assay, radioimmunoassay, sandwich assay, western blotting on polyacrylamide gel, immunoblotting, immuno-fluorescence assay, immunochemiluminescence assay or immunochromatography.

12. A method for diagnosing Alzheimer's disease by comparing a concentration of a β-amyloid antibody in a body fluid sample from a test subject with a concentration of a β-amyloid antibody in a body fluid sample from a control subject, both of which are measured by the method of claim 1.

13. A kit for diagnosing Alzheimer's disease, which comprises an antigen specific to a β-amyloid antibody and measures a concentration of the β-amyloid antibody through a reaction forming an antigen-antibody complex in a body fluid sample of a test subject.

14. The kit of claim 13, wherein the antigen is of a soluble, fibrillar, or aggregated β-amyloid.

15. The kit of claim 13, wherein the antigen is a fragment of Aβ1-42 protein represented by SEQ ID NO:3.

16. The kit of claim 13, wherein the fragment of Aβ1-42 protein is Aβ1-40 peptide or Aβ25-35 peptide.

17. The kit of claim 13, wherein the antigen forms a conjugate with streptoavidin or bovine serum albumin.

18. The kit of claim 13, wherein the body fluid sample is blood.

19. The kit of claim 13, which further comprises:
a reactor coated with the antigen specific to the β-amyloid antibody;
a secondary antibody-label conjugate detecting the antigen-antibody complex and comprising a label performing a chromogenic reaction by reacting with a chromogenic substrate;
a chromogenic substrate solution comprising the chromogenic substrate;
a washing solution used in each reaction step; and
a solution for stopping the chromogenic reaction.

20. The kit of claim 19, wherein the antigen is coated at a concentration in the range from 1 ng/100 μl to 100 μl/100 μl per the reactor.

21. The kit of claim 19, wherein the reactor is selected from the group consisting of a nitrocellulose membrane, a well plate made of polyvinyl resin, a well plate made of polystyrene resin, and a slide glass.

22. The kit of claim 19, wherein the label is selected from the group consisting of horseradish peroxidase, alkaline phosphatase, colloidal gold, fluorescein and dye.

23. The kit of claim 19, wherein the chromogenic substrate is selected from the group consisting of 3,3′,5,5′-tetramethyl bezidine, 2,2′-azino-bis(3-ethylbenzothiazoline-6-sulfonic acid) and o-phenylenediamine.

24. The kit of claim 19, wherein the antigen-antibody complex is detected by enzyme-linked immunosorbent assay, radioimmunoassay, sandwich assay, western blotting on polyacrylamide gel, immunoblotting, immuno-fluorescence assay, immunochemiluminescence assay or immunochromatography.


SEQUENCE LISTING (3 sequences)

SEQ ID NO: 1 - DNA, 3148 bp, Homo sapiens
  CDS (125)..(2377): amyloid A4 protein
gaattcccgcggagcagcgtgcgcggggccccgggagacggcggcggtagcggcgcgggcagagcaaggacgcggcggatcccactcgcacagcagcgcactcggtgccccgcgcagggtcgcgatgctgcccggtttggcactgctcctgctggccgcctggacggctcgggcgctggaggtacccactgatggtaatgctggcctgctggctgaaccccagattgccatgttctgtggcagactgaacatgcacatgaatgtccagaatgggaagtgggattcagatccatcagggaccaaaacctgcattgataccaaggaaggcatcctgcagtattgccaagaagtctaccctgaactgcagatcaccaatgtggtagaagccaaccaaccagtgaccatccagaactggtgcaagcggggccgcaagcagtgcaagacccatccccactttgtgattccctaccgctgcttagttggtgagtttgtaagtgatgcccttctcgttcctgacaagtgcaaattcttacaccaggagaggatggatgtttgcgaaactcatcttcactggcacaccgtcgccaaagagacatgcagtgagaagagtaccaacttgcatgactacggcatgttgctgccctgcggaattgacaagttccgaggggtagagtttgtgtgttgcccactggctgaagaaagtgacaatgtggattctgctgatgcggaggaggatgactcggatgtctggtggggcggagcagacacagactatgcagatgggagtgaagacaaagtagtagaagtagcagaggaggaagaagtggctgaggtggaagaagaagaagccgatgatgacgaggacgatgaggatggtgatgaggtagaggaagaggctgaggaaccctacgaagaagccacagagagaaccaccagcattgccaccaccaccaccaccaccacagagtctgtggaagaggtggttcgagaggtgtgctctgaacaagccgagacggggccgtgccgagcaatgatctcccgctggtactttgatgtgactgaagggaagtgtgccccattcttttacggcggatgtggcggcaaccggaacaactttgacacagaagagtactgcatggccgtgtgtggcagcgccattcctacaacagcagccagtacccctgatgccgttgacaagtatctcgagacacctggggatgagaatgaacatgcccatttccagaaagccaaagagaggcttgaggccaagcaccgagagagaatgtcccaggtcatgagagaatgggaagaggcagaacgtcaagcaaagaacttgcctaaagctgataagaaggcagttatccagcatttccaggagaaagtggaatctttggaacaggaagcagccaacgagagacagcagctggtggagacacacatggccagagtggaagccatgctcaatgaccgccgccgcctggccctggagaactacatcaccgctctgcaggctgttcctcctcggcctcgtcacgtgttcaatatgctaaagaagtatgtccgcgcagaacagaaggacagacagcacaccctaaagcatttcgagcatgtgcgcatggtggatcccaagaaagccgctcagatccggtcccaggttatgacacacctccgtgtgatttatgagcgcatgaatcagtctctctccctgctctacaacgtgcctgcagtggccgaggagattcaggatgaagttgatgagctgcttcagaaagagcaaaactattcagatgacgtcttggccaacatgattagtgaaccaaggatcagttacggaaacgatgctctcatgccatctttgaccgaaacgaaaaccaccgtggagctccttcccgtgaatggagagttcagcctggacgatctccagccgtggcattcttttggggctgactctgtgccagccaacacagaaaacgaagttgagcctgttgatgcccgccctgctgccgaccgaggactgaccactcgaccaggttctgggttgacaaatatcaagacggaggagatctctgaagtgaagatggatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactagacccccgccacagcagcctctgaagttggacagcaaaaccattgcttcactacccatcggtgtccatttatagaataatgtgggaagaaacaaacccgttttatgatttactcattatcgccttttgacagctgtgctgtaacacaagtagatgcctgaacttgaattaatccacacatcagtaatgtattctatctctctttacattttggtctctatactacattattaatgggttttgtgtactgtaaagaatttagctgtatcaaactagtgcatgaatagattctctcctgattatttatcacatagccccttagccagttgtatattattcttgtggtttgtgacccaattaagtcctactttacatatgctttaagaatcgatgggggatgcttcatgtgaacgtgggagttcagctgcttctcttgcctaagtattcctttcctgatcactatgcattttaaagttaaacatttttaagtatttcagatgctttagagagattttttttccatgactgcattttactgtacagattgctgcttctgctatatttgtgatataggaattaagaggatacacacgtttgtttcttcgtgcctgttttatgtgcacacattaggcattgagacttcaagcttttctttttttgtccacgtatctttgggtctttgataaagaaaagaatccctgttcattgtaagcacttttacggggcgggtggggaggggtgctctgctggtcttcaattaccaagaattc

SEQ ID NO: 2 - protein, 751 aa, Homo sapiens
MLPGLALLLLAAWTARALEVPTDGNAGLLAEPQIAMFCGRLNMHMNVQNGKWDSDPSGTKTCIDTKEGILQYCQEVYPELQITNVVEANQPVTIQNWCKRGRKQCKTHPHFVIPYRCLVGEFVSDALLVPDKCKFLHQERMDVCETHLHWHTVAKETCSEKSTNLHDYGMLLPCGIDKFRGVEFVCCPLAEESDNVDSADAEEDDSDVWWGGADTDYADGSEDKVVEVAEEEEVAEVEEEEADDDEDDEDGDEVEEEAEEPYEEATERTTSIATTTTTTTESVEEVVREVCSEQAETGPCRAMISRWYFDVTEGKCAPFFYGGCGGNRNNFDTEEYCMAVCGSAIPTTAASTPDAVDKYLETPGDENEHAHFQKAKERLEAKHRERMSQVMREWEEAERQAKNLPKADKKAVIQHFQEKVESLEQEAANERQQLVETHMARVEAMLNDRRRLALENYITALQAVPPRPRHVFNMLKKYVRAEQKDRQHTLKHFEHVRMVDPKKAAQIRSQVMTHLRVIYERMNQSLSLLYNVPAVAEEIQDEVDELLQKEQNYSDDVLANMISEPRISYGNDALMPSLTETKTTVELLPVNGEFSLDDLQPWHSFGADSVPANTENEVEPVDARPAADRGLTTRPGSGLTNIKTEEISEVKMDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - protein, 42 aa, Artificial [sequence omitted]